(19) United States
(12) Patent Application Publication
(10) Pub. No.: US 2016/0082130 A1
(43) Pub. Date: Mar. 24, 2016

(54) Dosage Forms and Methods for Diagnosing Sympathetic Nervous System Dysfunction

(71) Applicant: Francis B. Panosyan, Kingston (CA)

(72) Inventor: Francis B. Panosyan, Kingston (CA)

(21) Appl. No.: 14/810,842

(22) Filed: Jul. 28, 2015

Related U.S. Application Data
(60) Provisional application No. 62/029,668, filed on Jul. 28, 2014.

(30) Foreign Application Priority Data
Oct. 27, 2014 (CA) 2869060

(51) Int. Cl. A61K 49/00; A61B 5/00; A61B 3/11; A61K 9/00

(52) U.S. Cl. CPC A61K 49/0008; A61K 9/0048; A61B 5/4035; A61B 5/7271; A61B 3/112

(57) ABSTRACT

Described are methods for detecting and/or diagnosing systemic sympathetic nervous system dysfunction or disease in a subject. One embodiment comprises using a topical apraclonidine eye drop to detect an ocular condition indicative of sympathetic dysfunction or disease in a subject with Guillain-Barré syndrome (GBS). Embodiments can be used to detect and/or diagnose systemic sympathetic nervous system dysfunction or disease early, prior to development of substantial symptoms. Embodiments can also be used to unmask either bilateral ptosis or bilateral Horner's syndrome in GBS patients with the application of the drop to one eye. Also described are dosage forms suitable for use in the methods described herein. The dosage forms comprise an alpha receptor agonist with alpha-1 receptor activity. In one embodiment the dosage form comprises apraclonidine packaged as a single-use eye drop.

### FIELD

This invention relates to methods for diagnosing dysautonomia in a subject. More particularly, the invention relates to rapid, inexpensive, and non-invasive methods for diagnosing Guillain-Barré syndrome in a subject, and bilateral Horner's syndrome in a subject with Guillain-Barré syndrome. The invention also provides dosage forms of agents suitable for use in the diagnostic methods.

### BACKGROUND

Guillain-Barré syndrome (GBS) is a collection of clinical syndromes and the most common cause of acute flaccid paralysis in both adult and pediatric populations. The disorder affects the peripheral nervous system, typically characterized by ascending paralysis, weakness beginning in the feet and hands and migrating towards the trunk is the most typical symptom, with or without sensory disturbances, hyporeflexia or areflexia, a change in sensation or pain, as well as dysfunction of the autonomic nervous system. It can cause life-threatening complications, in particular if the respiratory muscles are affected or if the autonomic nervous system is involved. GBS is usually triggered by an infection, and the incidence is estimated to be 1.2-2.3/100 thousand with a mortality rate of 3-10%.

GBS remains a descriptive diagnosis for which there are no specific diagnostic tests. Clinical diagnosis is made based on a rapid development of the above-mentioned symptoms. Electrodiagnostic tests of nerves and muscles (such as nerve conduction studies) may also be employed in the diagnosis of GBS. In addition, cerebrospinal fluid analysis (through a lumbar spinal puncture) may provide evidence of CSF albumin-cytological dissociation, i.e., an elevated protein level (100-1000 mg/dl), without an accompanying increased cell count (i.e., absence of pleocytosis).

However, diagnosis based on the above-mentioned symptoms is only possible when GBS has substantially progressed, and other diagnostic procedures such as CSF analysis are invasive.

### SUMMARY

According to one aspect of the invention there is provided a method for detecting and/or diagnosing dysautonomia in the ocular sympathetic nervous system of a subject, comprising: obtaining one or more first measures of one or more features of the subject's eyes; applying an agent to one of the subject's eyes, wherein the agent is an alpha receptor agonist with alpha-1 receptor activity; obtaining one or more second measures of the one or more features of the subject's eyes; and comparing first and second measures of at least one feature of the subject's eyes; wherein a result of the comparing provides detection and/or diagnosis of the dysfunction in an ocular sympathetic nervous system of a subject.

In one embodiment, applying an agent comprises applying apraclonidine.

In one embodiment, the at least one feature of the subject's eyes comprises palpebral fissure height. In another embodiment, the at least one feature of the subject's eyes comprises pupillary diameter.

In one embodiment, the dysfunction in the ocular sympathetic nervous system of the subject comprises bilateral ptosis. In another embodiment, the dysfunction in the ocular sympathetic nervous system of the subject comprises bilateral ptosis; and the method includes diagnosing Guillain-Barré syndrome in the subject.

The comparing may include using a processor or a computer to analyze digital images of the subject's eyes and provide the one or more first measures of one or more features, the one or more second measures of the one or more features, and output the result of the comparison.

The result of the comparing may be indicative of dilation of a miotic pupil in a subject with Guillain-Barré syndrome.

In one embodiment, the method includes detecting and/or diagnosing bilateral Horner's syndrome in a subject with Guillain-Barré syndrome.

According to another aspect of the invention there is provided a dosage form of an agent for detecting and/or diagnosing a dysfunction in an ocular sympathetic nervous system of a subject, comprising: a dispenser that contains: a vehicle; and an alpha receptor agonist with alpha-1 receptor activity mixed with the vehicle; wherein the dispenser dispenses only a selected amount of the agent.

In one embodiment, the dosage form is adapted to dispense the agent to the eye of the subject.

In one embodiment, the dosage form is a single-use dosage form.

In one embodiment, the dispenser comprises an eye dropper.

In one embodiment, the alpha receptor agonist with alpha-1 receptor activity comprises apraclonidine. The apraclonidine may be provided at a dosage rate of 0.05%.

Another aspect of the invention relates to use of an agent to detect and/or diagnose dysautonomia in the ocular sympathetic nervous system of a subject, comprising: obtaining one or more first measures of one or more features of the subject's eyes; applying the agent to one of the subject's eyes, wherein the agent is an alpha receptor agonist with alpha-1 receptor activity; obtaining one or more second measures of the one or more features of the subject's eyes; and comparing first and second measures of at least one feature of the subject's eyes; wherein a result of the comparing provides detection and/or diagnosis of the dysfunction in an ocular sympathetic nervous system of a subject.

In one embodiment, the agent comprises apraclonidine.

In one embodiment, the at least one feature of the subject's eyes comprises palpebral fissure height. In another embodiment, the at least one feature of the subject's eyes comprises pupillary diameter.

In one embodiment, the dysfunction in the ocular sympathetic nervous system of the subject comprises bilateral ptosis. In another embodiment, the dysfunction in the ocular sympathetic nervous system of the subject comprises bilateral ptosis; and the use includes diagnosing Guillain-Barré syndrome in the subject.

The comparing may include using a processor or a computer to analyze digital images of the subject's eyes and provide the one or more first measures of one or more features, the one or more second measures of the one or more features, and output the result of the comparison.

The result of the comparing may be indicative of dilation of a miotic pupil in a subject with Guillain-Barré syndrome.

One embodiment relates to detecting and/or diagnosing bilateral Horner's syndrome in a subject with Guillain-Barré syndrome.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A and 1B are photographs of a patient with GBS (A) prior to treatment, and (B) after application of apraclonidine 0.05% to the left eye;

FIGS. 2A and 2B are photographs of another patient with GBS (A) prior to treatment, and (B) after application of apraclonidine 0.05%;

FIG. 3 is a graph showing percentage change in anisocoria and ptosis reversal after application of apraclonidine 0.05% to one eye, in five patients with a diagnosis of GBS and a group of nine control subjects; and

FIG. 4 is a graph showing percentage change in anisocoria after application of apraclonidine to one eye in five patients with a diagnosis of GBS and nine healthy subjects.

### DETAILED DESCRIPTION OF EMBODIMENTS

Described herein is a method of detecting and/or diagnosing disease, disorder, or malfunction of the autonomic nervous system (ANS), generally referred to as dysautonomia, that overcomes drawbacks of currently-used methods by being rapid, non-invasive, and able to detect/diagnose dysautonomia at a very early stage of progression. For example, the methods are applicable to the detection and diagnosis of conditions associated with and/or affecting blood pressure, cardiac function such as, e.g., postural orthostatic tachycardia syndrome (POTS), digestive tract peristalsis, sweating, and migraine. Using the methods described herein, dysautonomia may be detected or diagnosed before the substantial onset of one or more symptoms, and thus before widespread dysfunction of the autonomic nervous system.

For example, using the methods described herein, a disorder such as Guillain-Barré syndrome (GBS) may be detected in subjects prior to the substantial onset of one or more physical symptoms such as ascending paralysis, weakness beginning in the feet and hands and migrating towards the trunk, sensory disturbances, hyporeflexia or areflexia, a change in sensation or pain, as well as widespread dysfunction of the autonomic nervous system.

Autonomic dysfunction, in addition to respiratory compromise, is a known complication of GBS and it is a major contributor to mortality. There is interest in pupillary evaluation in general as a test for autonomic dysfunction (Bremner, F., Clin. Auton. Res. 19:88-101, 2009). Although bilateral ptosis has been reported in isolated case reports in patients with GBS (Ropper, A. H., Arch. Neurol. 43:1150-1152, 1986; Imam, Y. Z., et al., Case Rep. Neurol. Med. 2009, 2013(1365):178291; Pandey, S., et al., N. Z. Med. J. 125:78-79, 2012; Teng, H. W., et al., J. Emerg. Med. 43:e283-285, 2012), as such, bilateral ptosis remains an under-recognized feature of the disease. Furthermore, whereas there have been reports of bilateral ptosis in GBS cases, no diagnostic methods based on elucidating ptosis in GBS have been proposed.

Provided herein is a non-invasive method of reliably diagnosing GBS in a subject based on unmasking either bilateral ptosis or Horner's syndrome in a subject. The method includes administering an agent to one of the subject's eyes to determine involvement of the ocular sympathetic pathway as part of the diagnosis. Comparison of one or more features of the subject's eyes before and after administration of the agent yields an indication of a dysfunction in the sympathetic pathway, which provides a diagnostic of GBS in the subject.

Also provided herein is an agent in a suitable, sterile, single-use dosage form that can be used in accordance with the methods described herein. In one embodiment the dosage form comprises the agent packaged in a dispenser that allows a prescribed amount of the agent to be administered to the subject's eye. For example, the dosage form facilitates administration of the agent to the eye as a single drop, or as two drops, or the like. In one embodiment, the dosage form comprises a selected amount of the agent provided in an applicator such as an eye dropper.

The agent comprises an alpha receptor agonist with alpha-1 receptor activity. In the dosages (i.e., concentrations) administered in accordance with embodiments described herein, the agent has substantially no effect on pupils of healthy subjects. The agent may comprise an inactive component such as vehicle (e.g., an isotonic solution such as physiological saline) into which the alpha receptor agonist with which alpha-1 receptor activity is combined (e.g., diluted).

For example, the agent may comprise one or more of amidephrine, anisodamine, anisodine, apraclonidine, cirazoline, dipivefrine, dopamine, ephedrine, epinephrine (adrenaline), etilefrine, ethylnorepinephrine, 5-fluoronorepinephrine, 6-fluoronorepinephrine, indanidine, levonordefrin, metaraminol, methoxamine, methyldopa, midodrine, naphazoline, norepinephrine (noradrenaline), octopamine, oxymetazoline, phenylephrine, phenylpropanolamine, pseudoephedrine, synephrine, and tetrahydrozoline. Insofar as efficacy (i.e., selectivity) for the methods described herein might be enhanced by the agent's weak alpha-1 agonist activity; such activity may be optimized by appropriate known pharmacological approaches, such as dilution to a selected concentration, adjusting dosage rate, etc. This may be the case for agents with strong alpha-1 agonist activity, such as midodrine, epinephrine, and dopamine. For example, dopamine at different concentrations targets different adrenergic receptors.

In one embodiment, the agent comprises apraclonidine (also known as iopidine; 2,6-dichloro-N-(4,5-dihydro-1H-imidazol-2-yl)benzene-1,4-diamine). The apraclonidine may be administered at a dosage of, e.g., 0.01%-2%, or 0.05%-2%, or 0.05%-1%, although other dosages may be used. Smaller doses may be useful in disease selectivity. Apraclonidine is a sympathomimetic used in laser eye surgery (e.g., treatment of glaucoma), for prevention and treatment of postsurgical intraocular pressure elevation and for short-term adjunctive therapy in patients who require additional redirection of intraocular pressure. Apraclonidine is primarily an alpha-2 receptor agonist, but it also has a weak alpha-1 affinity. Apraclonidine has sensitivity and specificity in diagnosing Horner's syndrome related to structural lesion affecting the pupillary sympathetic dysfunction. The action of apraclonidine is thought to be due to denervation hypersensitivity of alpha-1 receptors in the pupil dilator muscle and Muller's muscle. Although apraclonidine has substantially no effect on healthy pupils, apraclonidine was tested on healthy subjects to quantify the sensitivity of the diagnostic, as described herein.

It is known that apraclonidine may be useful in the diagnosis of Horner's syndrome. In Horner's syndrome, the sympathetic innervation to the pupillary dilator muscle is lost. The affected pupil is thus miotic and the pupillary dilator responds to denervation by increasing alpha-1 receptors. When applied to the denervated (and thus hyper-sensitive) pupillary dilator muscle, a super-normal dilatory response is generated in which the pupil dilates to a degree greater than that which would be seen in a non-denervated muscle. This causes the reversal of anisocoria that is characteristic of Horner's.

The methods described herein also provide a diagnosis of bilateral Horner's syndrome in subjects with GBS, by providing evidence of pupillary sympathetic dysfunction. However, prior to this disclosure, bilateral Horner's syndrome was unknown as a manifestation of autonomic dysfunction in GBS. In one embodiment described herein, bilateral Horner's was unmasked by administration of apraclonidine to one eye, resulting in reversal of the miosis. Miosis reversal may be detected and/or quantified using any suitable technique, such as determining a ratio of change in anisocoria, or, at least in some cases, it may be identifiable through visual inspection. Involvement of the pupillary sympathetic nervous system may be correlated to a more severe disease process, likely due to the more extensive involvement of the sympathetic autonomic system.

The methods described herein may be either a valuable complement to routine investigations, and may help to confirm the diagnosis of GBS, or a stand-alone diagnostic for GBS. Unmasking bilateral ptosis according the methods described herein would be of great clinical value in accelerating further investigations to confirm the diagnosis and is expected to lead to earlier treatments. Furthermore, the methods described herein provide a simple, inexpensive, rapid, and objective test that can be performed without the need for extensive clinical experience.

Embodiments will be further described by way of the following non-limiting Example.

### Example

### Subjects

Patients with a diagnosis of GBS based on history and clinical exam were recruited for this study. Clinical profiles of the five patients are provided in Table 1. Patients with diabetes were excluded from the study. Patients on medications or eye drops with either sympathetic or parasympathetic action were also excluded from the study. All patients were examined by a neurologist, had an MRI of the spine and had CSF analysis at the time of their presentation. All patients had electrophysiological studies done in terms of nerve conduction studies at the time of their diagnosis. Patients on opioid therapy were not excluded. In total, five patients met the criteria for inclusion. Healthy control participants were recruited in outpatient clinics from partners and family members accompanying a person with an appointment. Participants with diabetes, ocular disease, previous ocular surgeries, and on sympathetic or parasympathetic medications or eye drops were also excluded from the study.

TABLE 1
Clinical profiles of subjects

| | Age | | | Deep | Time from | | | |
|---|---|---|---|---|---|---|---|---|
| | and | | Prodromal | Tendon | onset of | MRI of | | |
| Patient | Gender | Presenting Symptoms | Disease | Reflexes | Symptoms | Spine | CSF | NCS |
| A | 58 | Progressive symmetric | Deep venous | Areflexia | 7 days | Normal | No | Demyelinating |
| | Male | bilateral lower | thrombosis and | in lower | | | pieocytosis | pattern of motor |
| | | extremity ascending | pulmonary | extremities +1 in | | | and elevated | nerve conduction |
| | | paralysis associated | embolus | upper extremities | | | protein | studies with |
| | | with anaesthesia. | | | | | (2.79 g/L) | increased latency and |
| | | (MRC sum score | | | | | | significantly reduced |
| | | 32/60) | | | | | | velocities. Absent |
| | | | | | | | | sensory action |
| | | | | | | | | potentials in all |
| | | | | | | | | limbs. |
| | | | | | | | | Absent F-wave |
| | | | | | | | | response in his upper |
| | | | | | | | | and lower extremities |
| B | 20 | Acute onset | Aspiration | Global | 7 days | Normal | Traumatic | Sensory and motor |
| | Male | progressive generalized | pneumonia | areflexia | | | LP: WBC 65, | axonal pattern with |
| | | weakness and back | | | | | RBC 77, | secondary |
| | | pain. (MRC sum score | | | | | protein | demyelination. |
| | | 23/60) | | | | | (1.19 g/L) | Absent F-wave |
| | | | | | | | | response in upper and |
| | | | | | | | | lower extremities |
| C | 71 | Acute progressive | Campylobacter | Areflexia | 3 days | Meningeal | WBC 8, RBC | Normal nerve |
| | Female | bilateral lower | jejuni infection | in lower | | and | 1, Glu 3.3 | conduction studies. |
| | | extremity weakness. | | extremities +1 in | | lumbosacral | and protein | Normal F-wave |
| | | Severe pain lower back | | upper extremities | | root | (1.87) | responses in upper |
| | | and extremities. (MRC | | | | enhancement | | and lower |
| | | sum score 5 5/60) | | | | Bony mass | | extremities. |
| | | | | | | in S1 | | |
| | | | | | | vertebral | | |
| | | | | | | body | | |
| D | 24 | Acute progressive | Undifferenti- | Areflexia | 7 days | Increased | No | Demyelinating |
| | Female | weakness in upper and | ated diarrheal | | | enhancement | pleocytosis | pattern of motor |
| | | lower extremities | illness | | | of the cauda | and elevated | nerve conduction |
| | | (MRC sum score | | | | equina | protein | studies with |
| | | 52/60) | | | | | (0.58 g/L) | increased latency and |
| | | | | | | | | reduced amplitudes. |
| | | | | | | | | Absent or prolonged |
| | | | | | | | | F-wave response in |
| | | | | | | | | upper and lower |
| | | | | | | | | extremities |
| E | 65 | Acute progressive | Undifferenti- | Normal | 7 days | Increased | No | Prolonged F-wave |
| | Female | weakness and | ated laryngitis/ | reflexes | | enhaucement | pleocytosis | response |
| | | parestthesia affecting | bronchitis | except for | | of the cauda | and elevated | |
| | | limbs and face | | absent in | | equina | protein | |
| | | | | ankles | | | | |

### Methods

Digital photographs of the subjects' eyes were obtained prior to the application of apraclonidine. A small pre-measured reference was used at the time of photography to help quantify measurements. Apraclonidine (0.05% drop) was added to the left eye and a follow up photograph was obtained some time (19-52 min) after the application of the drop.

The digital photographs were analyzed with Image-J software (Schneider, C. A., et al., Nat. Methods 9:671-675, 2012) to determine pupillary diameter and palpebral fissure heights. Standard deviation of the measurements for error propagation was determined by repeated computer measurement of the pre-measured reference in a digital photograph. Palpebral fissure height was determined by measuring the vertical distance between the eyelids with the measurement going through the centre of each pupil. Pupillary diameter was determined with two measurements (horizontal and vertical) and an average denoted as OS or OD used for each pupil for further calculations. Pupillary response was quantified with the percentage ratio (D2−D1)/D1×100, where D1 and D2 represent the baseline anisocoria (D1=OS−OD) and the drug-induced anisocoria (D2=OS*−OD*) respectively.

### Results

Apraclonidine drop application unmasked bilateral ptosis in all five GBS patients. Apraclonidine did not have any significant effect on palpebral fissure height in normal individuals. Reversal of ptosis in GBS patients was apparent with visual inspection. For example, FIG. 1A is a representative photograph of one patient (patient D) with GBS prior to application of apraclonidine 0.05%. The photograph of FIG. 1B is the same patient after the application of apraclonidine to the left eye, showing reversal of the ptosis. This patient had no pupillary involvement and mild disease progress which did not require intubation or ventilatory support.

Patients with bilateral ptosis and no pupillary involvement after the apraclonidine test had milder disease phenotype (MRS sum score 52−55/60) and did not require intubation and ventilatory support during their disease process.

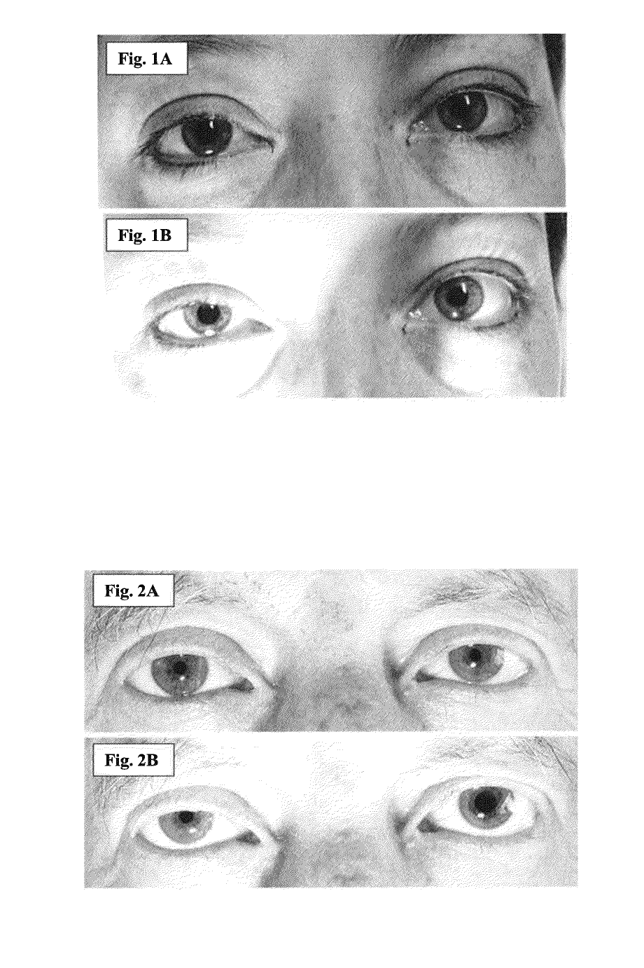

The test also unmasked bilateral Horner's syndrome with dilation of miotic pupil in two of the five patients with GBS. For example, FIG. 2A is a representative photograph of one patient with GBS prior to application of apraclonidine 0.05%. FIG. 2B shows the same patient after the application of apraclonidine showing reversal of the ptosis with improvement of the miosis in the left eye. This patient required intubation and ventilatory support.

Apraclonidine had no significant effect on pupillary diameter of healthy subjects. The patients with Horner's syndrome and pupillary involvement (miosis) had more severe disease phenotype both in terms of their overall weakness (MRS sum score 23−32/60) and respiratory deterioration which required intubation and ventilatory support.

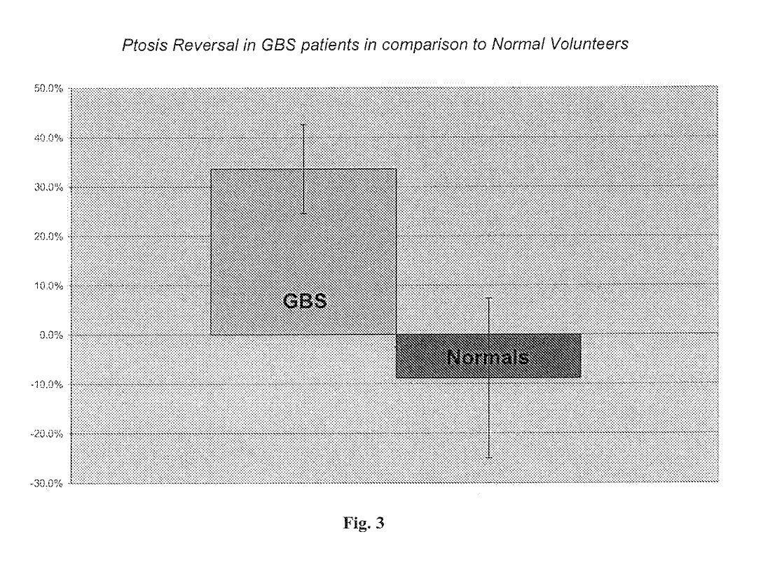

Computer analysis of the digital photographs in the patients with only ptosis showed an amplification of palpebral fissure height difference in the magnitude of 1130% (±80%) after application of apraclonidine. Similar analysis of pupillary diameter in the patients with pupillary dysfunction showed reversal of the ptosis and amplification of anisocoria after apraclonidine administration to 1124% (±324%). Results are presented graphically in FIG. 3, which shows percentage change in anisocoria and ptosis reversal after application of apraclonidine 0.05% to one eye in five patients with a diagnosis of GBS and a group of nine normal volunteers. Analysis of the effect of apraclonidine on the pupils of healthy subjects revealed aproclonidine had either no effect or caused paradoxical miosis of the pupil.

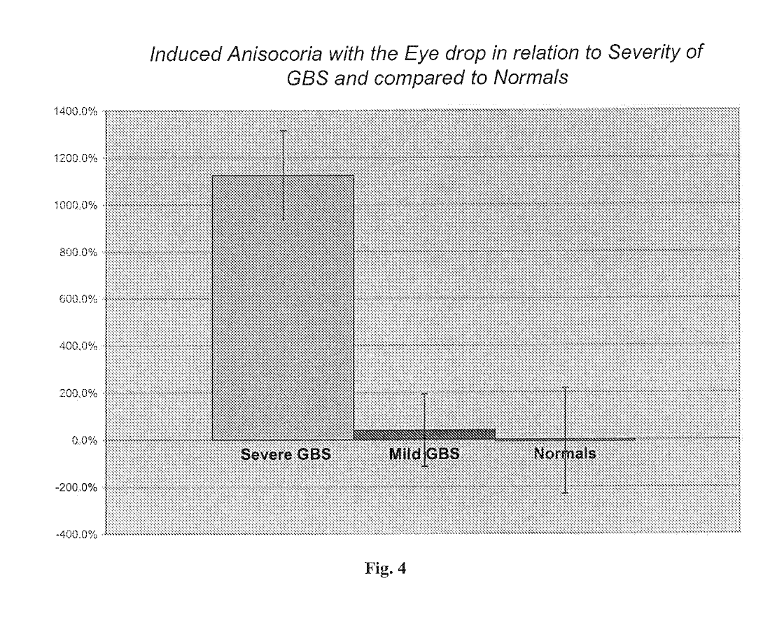

FIG. 4 is a graph showing percentage change in anisocoria after application of the eye drop to one eye in group of patients with a diagnosis of GBS and a group of normal volunteers. Patients with severe GBS required intubation for respiratory support (avg MRC sum score 28), patients with mild GBS did not require respiratory support (avg MRC sum score 55). Anisocoria was easily identified with visual inspection (see, e.g., FIG. 2).

### Discussion

The main diagnostic feature of GBS is rapidly progressive bilateral and relatively symmetric weakness of the limbs. The progression is typically within less than four weeks to clinical nadir and the vast majority of GBS patient become non ambulatory during their illness. The diagnosis of Guillain-Barré syndrome is challenging due to its many subtypes and the variability of the disease presentation, but global areflexia or hyporeflexia is often the single most useful feature of the disease that often prompts further investigations to help with the diagnosis in the emergency department.

The challenge in diagnosing GBS in patients is demonstrated by the heterogeneity and atypical features of the sample population herein. While patients A and D had typical GBS presentation and findings, including a demyelinating pattern of nerve damage, patient B had an axonal variant of the disease. Patient C had a bony lesion that was uncovered through MRI studies and did not have typical electrophysiological findings suggestive of GBS (Table 1). On the other hand, she had some typical features of the disease with a history of ascending paralysis preceded by paresthesias in her feet. She also had a preceding infection with Campylobacter jejuni which is strongly associated with GBS pathogenesis. Although there is some evidence in the literature of GBS presenting secondary to a neoplastic processes, this remains a controversial issue and is not widely accepted.

The atypical features seen in one of the GBS patients highlights the superior sensitivity of the apraclonidine test described herein. In fact, the sensitivity of the test is estimated to be better than 95% given the dichotomous categorical outcome of the test (presence of ptosis) where normal individuals had no significant response to apraclonidine.

Photographic evidence from the subjects in this study reveals that apraclonidine also unmasks a reverse ptosis of the lower eyelid. This observation supports the argument that the bilateral ptosis observed in GBS is related to sympathetic dysfunction. In reverse ptosis, the muscle of Riolan (which is located in the lower lid and is innervated by the sympathetics) seems to be affected by the disease process and contributes to the extent of bilateral ptosis.

In addition to the bilateral ptosis seen in GBS patients, there was clear evidence of pupillary sympathetic dysfunction and bilateral Horner's syndrome in some GBS patients. Bilateral Horner's syndrome is a previously unknown feature of the autonomic dysfunction in GBS. Bilateral Horner's was unmasked with apraclonidine applied to one eye with reversal of the miosis. The extent of miosis reversal was quantified as a ratio of change in anisocoria (FIG. 3), but this was also identifiable through visual inspection (FIG. 2). Involvement of the pupillary sympathetic nervous system correlated to more severe disease process, likely due to the more extensive involvement of the sympathetic autonomic system. Importantly, being on opioid therapy did not have a significant effect on the use of apraclonidine for diagnosing GBS.

This study did not investigate how early the ptosis or Horner's can manifest in GBS, as the earliest time frame where a patient was tested was within 72 hours. There is however some evidence that denervation hypersensitivity of alpha-1 receptors in the pupil dilator muscle can be unmasked with apraclonidine within three hours of an insult to the sympathetic nervous system.

All cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered merely exemplary and the invention is not to be limited thereby.

1. A method for detecting and/or diagnosing dysautonomia through the ocular sympathetic nervous system of a subject, comprising:
obtaining one or more first measures of one or more features of the subject's eyes;
applying an agent to one of the subject's eyes, wherein the agent is an alpha receptor agonist with alpha-1 receptor activity;
obtaining one or more second measures of the one or more features of the subject's eyes; and
comparing first and second measures of at least one feature of the subject's eyes;
wherein a result of the comparing provides detection and/or diagnosis of the dysfunction in an ocular sympathetic nervous system of a subject.

2. The method of claim 1, wherein applying an agent comprises applying apraclonidine.

3. The method of claim 1, wherein the at least one feature of the subject's eyes comprises palpebral fissure height.

4. The method of claim 1, wherein the at least one feature of the subject's eyes comprises pupillary diameter.

5. The method of claim 1, wherein the dysfunction in the ocular sympathetic nervous system of the subject comprises bilateral ptosis.

6. The method of claim 1, wherein:
the dysfunction in the ocular sympathetic nervous system of the subject comprises bilateral ptosis; and
the method includes diagnosing Guillain-Barré syndrome in the subject.

7. The method of claim 1, wherein the comparing is indicative of dilation of a miotic pupil in a subject with Guillain-Barré syndrome.

8. The method of claim 7, wherein the method includes detecting and/or diagnosing bilateral Horner's syndrome in a subject with Guillain-Barré syndrome.

9. A dosage form of an agent for detecting and/or diagnosing a dysfunction in an ocular sympathetic nervous system of a subject, comprising:
a dispenser that contains:
a vehicle; and
an alpha receptor agonist with alpha-1 receptor activity mixed with the vehicle;
wherein the dispenser dispenses only a selected amount of the agent.

10. The dosage form of claim 9, wherein the dosage form is adapted to dispense the agent to the eye of the subject.

11. The dosage form of claim 9, wherein the dosage form is a single-use dosage form.

12. The dosage form of claim 9, wherein the dispenser comprises an eye dropper.

13. The dosage form of claim 9, wherein the alpha receptor agonist with alpha-1 receptor activity comprises apraclonidine.

14. The dosage form of claim 13, wherein the apraclonidine is provided at a dosage rate of 0.05%.
